(19) United States
(12) Patent Application Publication
(10) Pub. No.: US 2023/0150616 A1
(43) Pub. Date: May 18, 2023

(54) Remote Controlled Aquatic Analytical and Sampling Apparatus with Bioremediation Capabilities

(71) Applicants: Nicolas Canello, Alta Loma, CA (US); Nidia Yamileth Lopez-Canello, Alta Loma, CA (US)

(72) Inventors: Nicolas Canello, Alta Loma, CA (US); Nidia Yamileth Lopez-Canello, Alta Loma, CA (US)

(21) Appl. No.: 17/528,162

(22) Filed: Nov. 16, 2021

(51) Int. Cl. B63B 35/00; G01N 1/10

(52) U.S. Cl. CPC B63B 35/00; G01N 1/10; B63B 2035/008; G01N 2001/1025

(57) ABSTRACT

The technology relates to an apparatus, methods and applications which take readings and samples in contaminated bodies of water at desired locations and depths. The apparatus is a remote control boat or similar apparatus that can travel on the surface, partially submersed or fully submersed. Onboard remote control instruments read pH, temperature, conductivity, salinity, turbidity, total dissolved solids, dissolved oxygen, oxidation-reduction, ammonia, nitrate, phosphate as well as algae and chlorophyll levels. It can also perform sludge blanket mapping and take water or sludge samples at different depths of the water column or at the bottom of the body of water. The data obtained permits to evaluate the health of a contaminated body of water to develop appropriate bioremediation procedures and monitor progress. The apparatus can also deliver the necessary bioremediation products. The danger for humans to navigate on contaminated waters is avoided with the remote control apparatus.

### BACKGROUND

In order to develop an effective bioremediation strategy for a contaminated body of water or to troubleshoot its problems, various parameters need to be measured. Additionally, samples of water and sludge need to be taken and analyzed in a laboratory. With the information collected from the analysis, a proper bioremediation process can be developed and the right bioremediation products can be applied. During the bioremediation process, more measurements and samples need to be collected to determine if the bioremediation process is working or if it needs to be modified. Normally, in order to measure these parameters, collect samples and apply bioremediation products, a person needs to get on a boat to do this. However, when the body of water is contaminated, it is dangerous for the person or persons on the boat. The danger may come from falling into contaminated waters with unknown contaminants or from the wind spraying contaminated water. Some possible contaminants can be pathogenic bacteria, parasites, cyanobacteria toxins or industrial toxins that have made it into the body of water. Additionally, contaminated bodies of water can produce gasses that affect the lungs, the brain and may cause death. Some such gases are ammonia and various sulfur compounds including hydrogen sulfide.

Some important parameters that often need to be measured are pH, water temperature, conductivity, salinity, turbidity, total dissolved solids, dissolved oxygen, oxidation-reduction, ammonia, nitrate, phosphate as well as algae and chlorophyll levels. Often, these parameters need to be evaluated at different locations and depths of the body of water to find the root cause or causes of the problems. In addition, it is often necessary to collect samples of water or sludge for further laboratory analysis. The samples would allow the identification of toxic chemicals, mineral contaminants and harmful biology. Additionally, micronutrient and macronutrient content analysis of the samples may reveal deficiencies which prevent the biology from consuming contaminants. Micronutrients cannot be evaluated with probes and need to be analyzed in a lab from the samples collected. For the same reason, the laboratory analysis of the samples would be necessary to determine if toxic heavy metals are present. It is also very important to take samples of water at various locations and depths to do laboratory work such as biological oxygen demand (also known as BOD₅) which is a measure of contamination. Different concentrations at different locations or depths may indicate problems with water flow patterns or contaminant runoffs. Once the root causes of the problems affecting the body of water are understood, a proper bioremediation process can be implemented to apply the right products to the body of water at the right locations.

Difference in parameter values at different locations and depths may reveal water and sludge stratification as well as problem areas where contaminants or harmful biology are building up. The samples of water and sludge provide valuable microbiological information such as types of harmful microorganisms and types of cyanobacteria that may be harmful to the body of water as well as to humans and animals. The data obtained is valuable to protect sources of fresh water and contaminated recreational bodies of water as well as to enhance the performance of wastewater lagoons or ponds.

For example, differences in water temperature in the same spot at different depths can reveal temperature stratification. Warmer water travels on the surface while colder denser water sinks to the bottom of a lagoon. This is one of the main causes of short circuiting in wastewater lagoons or ponds. A short circuit occurs when incoming wastewater is warmer than the water in the lagoon or pond. Consequently, the incoming wastewater does not travel throughout the entire volume of the lagoon or pond to get enough detention time for the contaminants to be degraded by the biology. Instead, it travels through a very small portion such as only on part of the surface of the lagoon or pond. Temperature stratification also causes reduced mixing of incoming warm surface water with bottom stagnant cold water. This situation creates a septic zone at the bottom with low or no dissolved oxygen. Because of the low water flow, this stagnate septic zone can have high biological oxygen demand (a measure of contamination) caused by the heavier organic particles depositing and building up at the bottom. Temperature readings at different depths and locations would reveal the actual flow of wastewater. This information would be vital to reduce short circuiting. The data would be very valuable to decide where best to place a curtain to break and disperse the water flow. It would also be very useful to position aerators in the lagoon at the right sites to prevent short circuiting and save energy by optimizing their locations.

It is also important to have the ability to take readings of dissolved oxygen and oxidation-reduction potential at various locations and depths. Low or absent dissolved oxygen or negative oxidation-reduction potentials can indicate the presence of “dead spots” which are areas where there is low water flow and sediment accumulation. Sometimes it may be necessary to submerge the probe in the sludge blanket layer at the bottom of the body of water to obtain data. If dissolved oxygen is absent, the sludge would be septic and potentially fertile ground for undesirable organisms such as filamentous bacteria and zooglea which can cause “bulking problems” (difficult-to-settle sludge which causes turbidity and high dissolved solids). Additionally, septicity promotes reproduction of sulfate reducing bacteria which convert sulfate into harmful hydrogen sulfide and other putrefaction components responsible for foul odors. Hydrogen sulfide is dangerous to humans, fish and animals. In addition, hydrogen sulfide has the ability to consume dissolved oxygen further reducing available oxygen in the body of water.

Total suspended solids and turbidity readings at various locations and depths are very useful to determine the settleability of suspended particles in different areas of the body of water. The data can help pinpoint areas where the biology is not properly cleaning contaminants in wastewater lagoons, ponds or in contaminated recreational bodies of water. This data is vital for troubleshooting and to take proactive measures to enhance the settleability and degradability of dissolved solids before the body of water becomes overwhelmed by contaminants and deposits. If this situation occurs, the body of water and its effluent would become turbid with high biological oxygen demand.

Readings of pH are essential to assess the health of the biology of a body of water. Once again, the pH probe may need to take readings in the water column or may need to be inserted inside the sludge blanket to get sufficient data. If the pH is low, it indicates the likelihood of harmful fungi or yeast growing as well as the possibility of harmful acidic contaminants. If the pH is too high, it may indicate excessive algae or unexpected alkaline contaminants in the influent. pH that is too low or high (below 6 or above 8) drastically reduces the ability of the biology in the body of water to digest contaminants. Changes in pH at different depths may spot unforeseen issues before they become serious problems such as fertilizer or chemical runoffs, incoming contaminants or areas where harmful biology may be growing.

There are algae monitoring probes that use fluorimetry to evaluate the concentration of algae and chlorophyll in order to monitor algae blooms. This is valuable to determine when a body of water is in the early stages of eutrophication or to troubleshoot it if it is already eutrophied. Excess algae are responsible for several problems. Dead algae cause oxygen depletion causing the death of fish as well as the production of hydrogen sulfide and other putrefaction malodors. Furthermore, excess algae can significantly affect the attractiveness of a recreational body of water. The ability to check the levels of algae and chlorophyll at different locations and at different depths can serve as an early warning system to develop proactive measures and prevent expensive consequences. It can also show locations where the algae levels are higher and pinpoint potential root causes such as nutrient (nitrogen and phosphorous) runoffs. Some species of algae produce cyanotoxins which are substances highly toxic to fish, humans or animals. However, for proper identification of cyanobacteria that produce toxins, sampling of water and laboratory analysis are needed to identify the toxic species.

Sludge buildup reduces the volume of a body of water reducing the time that contaminated water remains in a wastewater lagoon or pond or a contaminated body of water with water flowing in and out of it. The biology in the system needs enough detention time to digest the contaminants. High sludge levels also create channels where wastewater flows around causing short circuits. In wastewater lagoons or ponds and contaminated recreational bodies of water, high sludge levels can be very detrimental for the health of humans, animals and fish. Mapping the thickness and distribution of the sludge blanket in the body of water is important to target the application of bioremediation products to enhance the digestion of sludge. Areas with a thicker sludge blanket at the bottom may require higher amounts of bioremediation substances.

Sludge blanket mapping is necessary before the bioremediation process and during the process to evaluate the effectiveness of the bioremediation products used to digest the sludge blanket. Monitoring the sludge blanket thickness is important to determine if the bioremediation products are working or if a change in strategy is necessary. A sonar system as described in this patent application can be carried by the remote control apparatus and map the sludge blanket thickness throughout a body of water. This can be done safely even in heavily contaminated wastewater lagoons, ponds and contaminated recreational bodies of water because there is no need for humans to enter the contaminated environment.

In wastewater lagoons or ponds, water samples are useful for biological analysis such as evaluation of floc structure and higher lifeforms (protozoa and metazoa) analysis. This information reveals sludge age (degree of contaminant degradation) and the presence of harmful organisms (excess filamentous bacteria, fungi, yeast, alga, septic bacteria, toxic cyanobacteria or zooglea). The identification of these organisms often exposes root causes of problems such as septicity, nutrient imbalances (nitrogen and phosphorous), poor food to biomass ratio as well as the presence of fat, oil and grease. Besides water samples, it is important to take samples of the sludge blanket at the bottom of the body of water. The thickness of the sludge blanket normally varies at different locations depending on water flows, currents or the density of the various deposits such as sand or precipitated minerals. Samples of the sludge blanket provide valuable mineral and microbiological data that can be used to decide on the optimum application points for bioremediation. In addition, the sludge samples can be used to determine the percentage of volatile solids in the sludge which is an indication of its degree of degradation. This information indicates if the sludge is being properly degraded or if it is poorly digested exposing weaknesses in the biology of the body of water. Furthermore, the sludge sample can be analyzed to determine its organic composition such as types of fibers or other organic substances in order to customize a bioremediation program to degrade those organic components.

If the body of water is contaminated, a person on a boat is exposed to the water as well as toxic gases that may come from it. There is a need for a remote control apparatus that can enter contaminated bodies of water and take multiple readings of the necessary parameters at various locations and depths. There is also a need for such apparatus to take water and sludge samples for chemical and microbiological laboratory analysis. Once the data from readings and laboratory analysis has been evaluated, a customized bioremediation process can be developed. The same apparatus can safely apply the bioremediation products at the proper locations and depths to maximize their effectiveness.

### BRIEF SUMMARY

The present patent application refers to a remote control apparatus such as a boat or similar surface, partially submersed or fully submersed apparatus. For simplicity, the apparatus is referred here as “boat”. The boat can carry instruments that take measurements of various parameters at different depths to assess the health of a body of water. The apparatus can also take water and sludge samples as well as apply the necessary bioremediation products. The body of water can be contaminated recreational bodies of water and wastewater lagoons or ponds.

A body of water can experience a wide range of problems such as high turbidity, floating solids, malodors, excess algae, excess plant growth, death of fish, death of wild life, high coliform bacteria counts, excess biological oxygen demand (BOD₅), high ammonia or nitrates, high salinity, high or low pH and others. In order to determine the root cause or causes of problems, multiple parameters need to be measured. Some of these parameters are pH, water temperature, conductivity, salinity, turbidity, total dissolved solids, dissolved oxygen, oxidation-reduction, ammonia, nitrate, phosphate as well as algae and chlorophyll levels. Often, the sludge blanket may need to be penetrated to read pH, dissolved oxygen and oxidation-reduction potential. In addition, it is often necessary to collect samples of water and of sludge from the body of water. These samples can be used to do laboratory mineral analysis. If levels of some minerals are too low, they can limit the biology in the body of water to digest contaminants efficiently. The analysis may also reveal the presence of toxic heavy metals (chromium, lead, mercury, cadmium, arsenic, nickel, etc.) at levels that are not acceptable. The same water and sludge samples can be used to do biological analysis that could show the presence of coliforms, specific pathogens, toxic cyanobacteria or problematic microorganisms such as filamentous bacteria or zooglea. The analysis may also reveal microorganisms associated with septicity, low pH, poor levels of nutrients (nitrogen and phosphorous) as well as the presence of grease and oils.

Normally, in order to take readings to evaluate the parameters described earlier or to get water or sludge samples, a person or persons need to get on a boat. Then, they travel from location to location to take such readings and collect samples. If the body of water is contaminated, there are serious dangers such as toxins from organic or chemical sources as well as cyanotoxins produced by some algae species. Pathogens can also be present in the form of bacteria, protozoa or parasites. Some of the dangers of getting on a boat in contaminated waters are spray from wind, falling overboard or toxic gases emanating from the body of water. Some of these gasses are hydrogen sulfide, other sulfur compounds, ammonia and unknown gasses depending on the type of contamination in the body of water.

A remote control apparatus or boat that can be driven to the desired locations to take readings and samples would be a safe alternative. The boat can be driven with a commercially available long range transmitter (44, FIG. 1 and FIG. 2) and receivers in the remote control elements of the boat. The same unit can control the other remote controlled elements described in this patent application. A commercially available remote controlled servo (24, FIG. 4) can regulate the speed of the motor to control the speed of the boat. In order to control the direction of the boat, a remote controlled servo (25, FIG. 4) can control rudders (26, FIG. 4) via an attached rod (27, FIG. 4). The maximum operational distance would be determined by selecting the appropriate transmitter (44, FIG. 1 and FIG. 2) and receivers in the remote controlled elements in the boat.

The boat can carry various probes or a multi-probe (2, FIG. 1) that can read various parameters simultaneously. There are commercially available multi-probe systems with telemetry and with a pressure sensor (8, FIG. 1, FIG. 2 and FIG. 7) incorporated to measure the depth where the readings are taken. The readings are taken at any point of the water column and when needed into the sludge blanket at the bottom of the body of water. The probe is lowered by a remote control reel on the boat utilizing a transmitter (44, FIG. 1 and FIG. 2) and a receiver on a servo that controls the reel.

A depth sensor such as a water pressure sensor with telemetry can determine when the desired depth has been reached. The servo-operated reel can also retrieve the probe when the data is collected. A sludge sample or water sample can be collected with a bacon bomb. This is a device that is normally used to collect water or sediment samples at the bottom of tanks or petroleum and gasoline containers. The bacon bomb hits the bottom and a plunger assembly opens to admit the material. The plunger closes again when the bacon bomb is withdrawn, forming a tight seal. One or more remote control reels on the boat can be used if more than one sample is desired. Alternatively, a hose can be used and be reeled down to the desired depth to obtain the sample. The hose would be attached to a remote controlled self-priming pump on the boat that can be used to withdraw water or sludge samples at any depth of the water column. The hose would have a check valve (19, FIG. 2) near the opening of the sample-withdrawing end. The check valve prevents liquid from entering the hose as it is lowered until the sample is withdrawn. When the pump is engaged, the pump suction overcomes the check valve and withdraws the sample in the direction that the valve is designed to open. When the pump stops, the valve closes keeping the sample in the hose. The sample can be emptied into an onboard sample container or it can be kept in the hose to be withdrawn at shore. If excess sample is accidentally withdrawn, the pump can allow the sample container on board to overflow with an opening toward the body of water. If the sample is to be kept inside the hose, the exit point of the pump can overflow the excess sample into the body of water. Additionally, a remote controlled bladder pump can be used to collect samples. This is a non-contact, positive displacement, pneumatically operated pump that is normally used to collect ground water samples. Other similar water or sludge sampling devices such as a modified sludge judge and others can also be used.

Very often, it is necessary to determine the thickness of sludge and sediments in a body of water. Excess sludge blanket sediments are fertile ground of septic harmful biology such as pathogens, hydrogen sulfide producing bacteria and others. The sludge blanket itself also reduces the volume of the body of water. This is especially important in a wastewater plant if excess sludge volume reduces the time that wastewater remains in the lagoon or pond for biological degradation of contaminants. It is important to map the thickness of the sludge blanket at various points in the body of water to determine where to apply bioremediation products to digest the sludge blanket. Additionally, mapping of the sludge blankets is helpful to determine if a bioremediation program is working satisfactorily or if the bioremediation strategy needs to be changed. The boat can carry a sonar fish finder to map the thickness of sludge blanket. There are various commercially available models with mapping ability and with the necessary frequency to detect sludge sediments. The mapping data can be collected on a memory device on board or it can be sent via telemetry to shore where it is collected with a receiver unit with interface.

Once the root causes of the problems of the body of water have been established, a bioremediation program can be designed with the right products. Many known technologies used in the art of bioremediation are bacteria, fungi, actinomyces, streptomyces, enzymes and appropriate protozoa or metazoa. Other technologies used are biostimulants for microorganisms to help boost their biology. Some of these products are based on micronutrients and on plant extracts. Additional bioremediation technologies are chemicals that provide oxygen to septic areas such as sodium perborate, calcium peroxide, magnesium peroxide, calcium nitrate, potassium nitrate and various nitrate containing chemicals. Sometimes, chemicals are used to precipitate excessive amounts of phosphates that cause eutrophication. Some of these chemicals are aluminum salts, ferrous salts, ferric salts and lanthanum salts. The remediation product can be applied from the hull of the boat or it can be pumped from shore.

### BRIEF EXPLANATION OF THE DRAWINGS

FIG. 1 Remote Control Boat with Probes to Evaluate the Health of the Body of Water.

FIG. 2 Remote Control Boat with Sampling Devices to Collect Water and Sludge Samples.

FIG. 3 Remote Control Boat with Bioremediation Capabilities.

FIG. 4 Remote Control Boat with Elements that Regulate Speed and Direction.

FIG. 5 Side View of Plate Attachment to the Boat with Connector Tube and Swivel.

FIG. 6 Back View of Boat with Plate Attachment, Connector Tubes, Swivels, Remediation Double Hose and Application Double Hose.

FIG. 7 Remote Control Boat with Double Hoses Feeding Bioremediation Product and Air.

### DETAILED DESCRIPTION

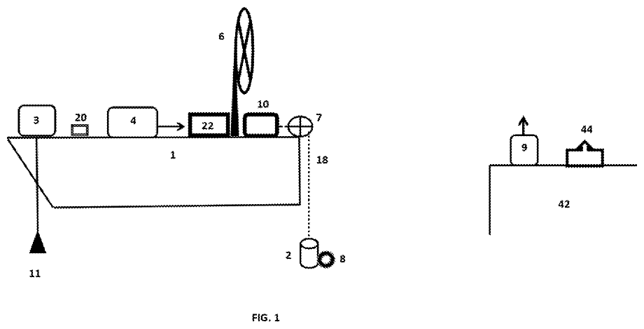

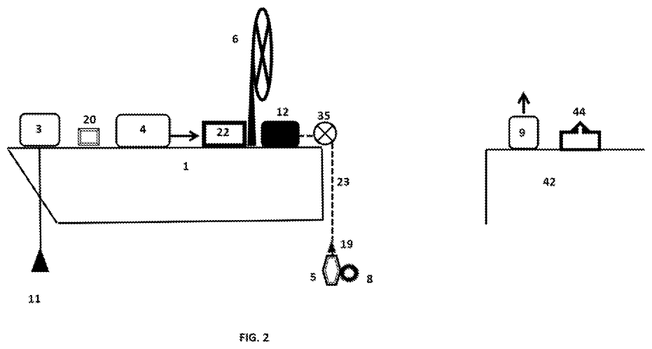

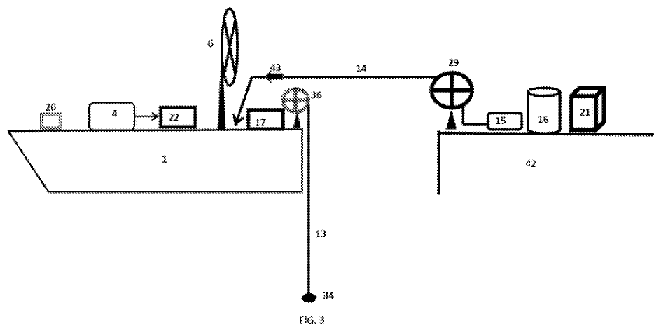

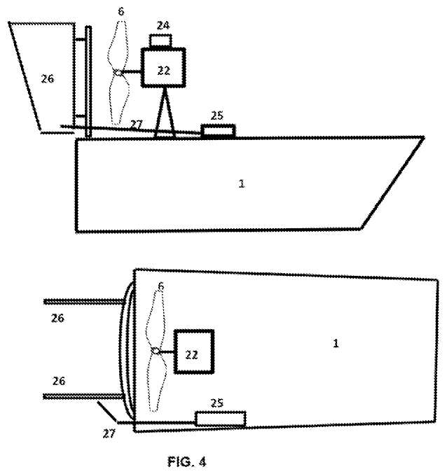

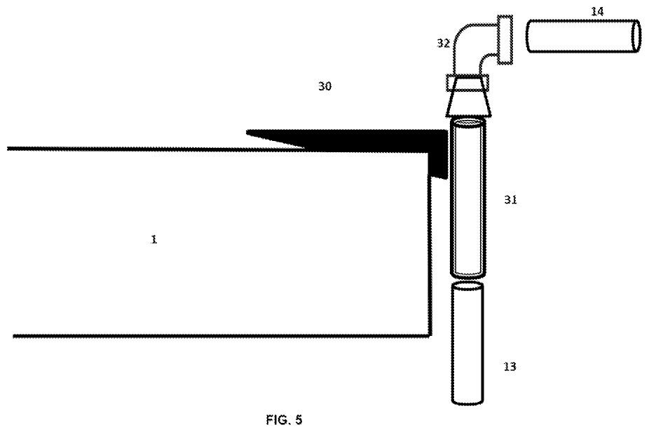

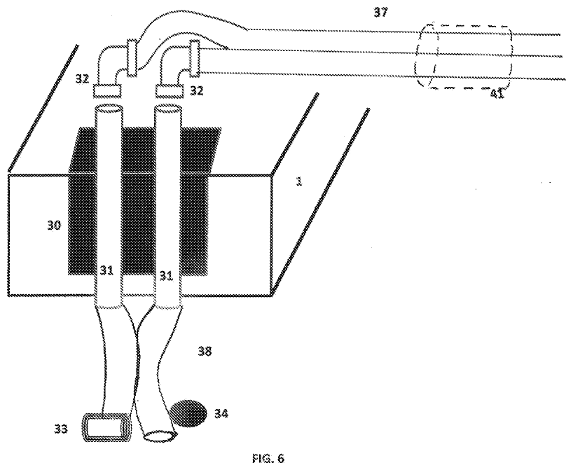

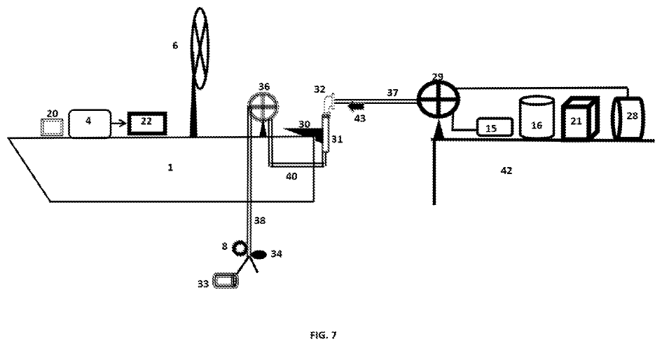

The present patent application refers to a remote control apparatus such as a boat or a similar surface, partially submersed or fully submersed apparatus. The apparatus is driven from shore using a commercially available long-range transmitter (44, FIG. 1 and FIG. 2) and receivers on the remote controlled elements on the boat. A remote controlled servo (24, FIG. 4) on the apparatus regulates the speed of the motor (22, FIG. 1, FIG. 2, FIG. 3 and FIG. 4) while another remote control servo (25, FIG. 4) adjusts the rudders (26, FIG. 4) to control the direction. The length or the width of the apparatus can range from 1 foot to 30 feet. For simplicity, the apparatus here described is referred to as “boat” (1, FIG. 1, FIG. 2, FIG. 3, FIG. 4, FIG. 5, FIG. 6 and FIG. 7). The power source (4, FIG. 1, FIG. 2 and FIG. 3) can be gas, gas mixed with oil, batteries, solar panels, a gas-driven electrical generator or an electrical marine cable or other suitable cable from the shore of the body of water. The apparatus can have one of various propelling configurations such as a propeller-driven boat, a water-jet propelled boat or one of any configuration or shape that allows it to travel on water, partially submersed in water or underwater. Some of the preferred configurations are those that reduce the chance of the apparatus getting tangled in roots or vegetation such as an airboat with air propeller blades (6, FIG. 1, FIG. 2, FIG. 3, FIG. 4 and FIG. 7).

The apparatus and methods of the patent application allow the safe evaluation of the health of bodies of water by taking readings, samples and mapping sludge sediments via remote control. It is dangerous for a person to get on a boat in contaminated bodies of water because wind can spray water, the person can fall overboard or because toxic gasses may emanate from the body of water. Once the root problems affecting the body of water have been determined, a bioremediation strategy can be developed. The boat has the capability to apply such remediation products.

The boat uses several probes or a multi-parameter probe (2, FIG. 1) to take readings at any depth of the water column or into the sludge blanket at the bottom of the body of water. Some of these parameters that can be read are pH, temperature, conductivity, salinity, turbidity, total dissolved solids, dissolved oxygen, oxidation-reduction, ammonia, nitrate, phosphorous as well as algae and chlorophyll levels. The metering devices and receivers of the remote controlled elements are protected by an enclosure (10, FIG. 1) or a low-weight roof to prevent damage from the rain or water sprayed by the wind. The probe is reeled down with a remote-control reel (7, FIG. 1) on the boat. The metering device connects to the multiparameter-probe or probes (2, FIG. 1) via a cable (18, FIG. 1). The data from the probe is either stored on an onboard memory device on the boat or it is transmitted to a receiver with visual interface (9, FIG. 1 and FIG. 2) at shore (42, FIG. 1, FIG. 2, FIG. 3, FIG. 7) via telemetry. The data would include the location and depth where the reading is taken using the GPS (20, FIG. 1, FIG. 2 and FIG. 3) on the boat which transmits its location via telemetry. The depths where the readings are taken are obtained from a pressure sensor (8, FIG. 1 and FIG. 2) with telemetry. The probes can take and send continual readings or they can be turned on only at the desirable locations. The data is streamed in real-time without having to return to shore to read it. In this manner, the apparatus monitors the different parameters at different locations and depths to find trouble spots and get the needed data and samples to troubleshoot the body of water. The receiver display interface can be a smart phone, tablet, laptop or other suitable platform that is compatible with the telemetry system.

There are companies such as In-situ in the United Kingdom that sell multi-parameter devices with the option of telemetry. In addition, a telemetry system can be designed where the probes are connected via their BNC connectors to an electrode amplifier and to an interface. The amplifier increases the voltage produced by the electrode probe into a range where it can be monitored by an interface device. A transmitter-receiver module streams data wirelessly in real-time at standard frequencies used in commercial systems. The telemetry can be any of the commercially available ethernets, GOES satellite, spread-spectrum radio etc. The interface can have multiple channels to monitor more than one device at the same time.

The apparatus can also do sludge blanket mapping using a fish-finder sonar. The sonar (3, FIG. 1 and FIG. 2) on board has telemetry capability and it can map the thickness of the blanket of sludge that builds up at the bottom of wastewater treatment lagoons or ponds, contaminated recreational bodies of water. The sonar is one of various commercially available models with mapping capability and with the necessary frequency to detect sludge sediments. The sludge blanket thickness is measured before bioremediation begins and can be measured during the bioremediation procedure to determine its effectiveness or if a different bioremediation approach is needed to better digest the sludge blanket.

The sonar transducer (11, FIG. 1 and FIG. 2) sends and receives sound waves. The signals are sent to the sonar unit (3, FIG. 1 and FIG. 2) which uses telemetry to transmit the data to shore. At shore, the data is captured by a receiver unit with a visual interface (9, FIG. 1 and FIG. 2). The data can also be stored in an onboard interface storage device to be read later. The sonar reads sludge levels by transmitting pulses of sound energy into the water and detecting the sound energy reflected back to the transducer (11, FIG. 1. and FIG. 2). The time that is required for the reflected sound energy to return to the transducer correlates with the depth of the reflecting object. The sludge blanket can be distinguished from the lagoon bottom by the difference in density. The difference in density is viewed in the image as difference in colors. Numerical data of depths is also provided by the sonar system. Typical transducers range in frequency from 50 kHz to 800 kHz. In order to map sediment sludge in the shallow waters of wastewater ponds or lagoons (20 feet or less), a high frequency is needed. Higher frequencies have shorter wavelengths and more wave cycles per second. This provides a higher definition picture that can map the sludge blanket thickness in spite of its low density and differentiate it from the higher density of the bottom of the body of water. In this application, frequencies of 400 kHz to 800 kHz can be used. The thickness and distribution of the sludge blanket throughout the body of water is essential to apply bioremediation products at the proper locations for its digestion. In addition to the ability to map sludge, the volume of the sludge blanket throughout the body of water can be calculated using commercial software (Biobase in Minneapolis). This is useful to monitor and evaluate the degradation of sludge using biological bioremediation products in lagoons or ponds. Such process is known in the industry as bio-dredging.

In deep water (over 20 ft.), a lower frequency transducer such as one lower than 200 kHz is normally used to map sludge at the bottom of contaminated recreational bodies of water or others. This allows for deeper penetration of the signal into the water. The signal sent from the sonar is projected downward as a “cone”. A sonar beam's cone angle indicates the extent to which a transmitted beam “spreads out” as it travels through the water column. The area covered at the bottom of the cone depends on the angle of the signal and the depth of the body of water. For shallow water, a wider cone angle is commonly used to make the area under observation large enough to be useful. However, if the angle is too wide, the signal is more susceptible to being scattered in an unproductive way by small objects in the water. In order to map sludge in the shallow water of wastewater plants (20 feet or less), lagoons or ponds, a narrow cone angle of 8 degrees to 20 degrees is useful to achieve higher resolution by concentrating the signal on a small area at the base of the cone. For deeper water, a small cone angle (8 degrees or less) is used because the distance traveled in the water column is longer increasing the area at the base of the cone at the bottom of the body of water. The smaller cone angle is necessary to avoid loss of resolution in the wider area of deep lagoons or ponds.

A multiple frequency sonar can also be used. Different frequencies reveal different levels of detail. For example, a high frequency pulse provides excellent detail, but cannot penetrate very deep into the water. A low-frequency 50 kHz pulse penetrates deeper, but reveals less detail. A multiple frequency transducer has a broad band system that uses multiple frequencies at the same time improving resolution. CHIRP (an acronym for Compressed High Intensity Radiated Pulse) is such a system which generates an image using a wider range of frequencies allowing the processor to produce a much more accurate and detailed sonar image at different depths. Lowrance sells fish-finder sonar systems with the right frequency and cone angle that allows the measurement of the sludge blanket in wastewater bodies which normally have a water column of 20 feet or less. Lowrance also has units that can map sludge at deeper depths.

The remote-control boat can take water or sludge samples. The samples can be taken at different depths of the water column or at the bottom of the body of water for further laboratory analysis. The location where the sample is taken is monitored using the onboard GPS (20, FIG. 1, FIG. 2 and FIG. 3) with telemetry and the depth where the sample is taken is recorded by the pressure sensor (8, FIG. 1, FIG. 2 and FIG. 7) also with telemetry capabilities. The body of water can be a contaminated recreational body of water or wastewater lagoon or pond. The sample can be obtained using a “bacon bomb” sampler (5, FIG. 2) which is a device commonly used to take samples from the bottom of tanks or petroleum and gasoline containers. The bacon bomb can be lowered using an on board remote controlled reel (7, FIG. 1) similar to the one used to lower the probes or it can use a hose reel (35, FIG. 2.) for the same purpose. The bacon bomb (5, FIG. 2) hits the bottom and a plunger assembly opens to admit the material. The plunger closes again when the bacon bomb is withdrawn, forming a tight seal. The boat can carry several remote controlled reels (7, FIG. 1) to lower the probe or probes and remote controlled reels (35, FIG. 2) to lower the sampling devices.

A hose can also be used and be reeled down from the boat to the desired depth using a remote-controlled hose reel (35, FIG. 2) previously described. The hose can be used to obtain a sample. If a hose is used to get the sample, the hose would be attached to a remote controlled self-priming pump (12, FIG. 2) on the boat which would withdraw water from any point in the water column or sludge from the sludge blanket. The sample hose (23, FIG. 2) would have a check valve (19, FIG. 2) at its submerged end to prevent liquid from entering the hose as the hose is reeled down. When the pump is engaged, the pump suction overcomes the check valve and withdraws the sample in the direction that the valve is designed to do. When the pump stops, the valve closes keeping the sample in the hose. The sample can be emptied into an onboard container or it can be kept in the hose and be withdrawn at shore. When the suction pump stops, the check valve closes and keeps the sample inside the hose. Alternatively, a bladder pump system can be used to get a sample. These types of pumps are commonly used to collect ground water samples. The pump can be controlled via remote control from shore and the sample can be collected on an onboard container on the boat. Other similar devices to take water or sludge samples can also be used such as a modified sludge judge or other.

Several remote control reels on the boat can be used to obtain various samples via “bacon bombs”, hoses or bladder pumps. The operator can keep track of where the samples were taken by recording the GPS locations. The pump or bacon bomb that holds any of the samples can be easily determined by which reel or pump is activated when the sample is taken. As an alternative to a pressure sensor to determine depth, the depth can also be approximated counting the turns of the reel and using the average diameter to determine the number of circumferences to approximate depth. The turns can be counted with an infrared sensor as well as a mechanical or digital tachometer that streams the data to shore in order to stop the reel when the desired depth is reached.

The reel can be a small commercially available remote control unwind and rewind reel sold by various distributors such as Pacific Marine & Industrial in Richmond, Calif. A reel can also be designed with a DC motor or servo that has the ability to reverse polarity to wind and unwind. The reel would lower the probe with an appropriate cable with BNC connectors compatible with the metering probe or sensors. A similar remote controlled reel or reels can be used to lower the sampler device or the sampling hose previously described as well as the application hose (13, FIG. 1).

The boat can carry a delivery system to apply bioremediation products to degrade contaminants, digest sludge, control odor, control excess algae, reduce coliform counts, eliminate toxicity and improve the overall performance and health of the body of water. In one configuration, the bioremediation products can be carried in the hull of the boat. A self-priming pump on the boat (17, FIG. 3) sends the bioremediation product via an application hose (13, FIG. 3) that can be lowered with an onboard reel (36, FIG. 3) or just hang at the depth where the product needs to be applied. The application hose (13, FIG. 3) carries a plastic weight (34, FIG. 3) at its end to keep the hose extended at its full length. Alternatively, the boat can be connected to shore (42, FIG. 3) via a bioremediation hose (14, FIG. 3). The bioremediation hose can refill the hull of the boat so that it does not need to return to shore. On shore, the bioremediation hose is connected to an on shore reel (29, FIG. 3). The reel in turn is connected to a pump (15, FIG. 3) which draws the bioremediation product from a large tank (16, FIG. 3) or from an onsite bioreactor (21, FIG. 3). The onsite bioreactor can be one such as U.S. Pat. No. 10,981,818 B2 which would grow microorganisms on site and apply them at the end of the fermentation cycle when their numbers are the highest. The boat can carry remote controlled reels for the probes (7, FIG. 1), for sampling devices (35, FIG. 2.) and to lower the bioremediation hose (36, FIG. 3).

On a different configuration, the bioremediation hose (14, FIG. 5) can bring the bioremediation product from shore and apply it directly to the body of water without the need of a remote controlled reel on the boat. In this configuration, the hull does not need to carry the bioremediation product to reduce the weight and drag on the boat motor. For this purpose, the boat would have a plate (30, FIG. 5) made of corrosion resistant material such as stainless steel or other. The plates have corrosive resistant connector tubes (31, FIG. 5) welded to it. On the top of each connector tube there is a swivel (32, FIG. 5) where the bioremediation hose (14, FIG. 5) from shore connects. The swivel connected to the hose reduces the chance of the bioremediation hose from tangling as the boat changes direction. On the lower part of the connector tube, the application hose (13, FIG. 5) can be connected to deliver the bioremediation product. The length of the application hose can be selected to apply the bioremediation product at the bottom of the body of water under the sludge blanket or at any point in the water column. A plastic weight at the end of the hose ensures that the application hose is extended to the full length. When the bioremediation hose applies the bioremediation product from the pump on shore (15, FIG. 3), it is desirable that the application stops when the pump on shore is turned off. In this manner, bioremediation product is not wasted as the boat moves to a new location for product application. In order to stop application when the pump at shore is turned off, a one-way valve (43, FIG. 3) is placed on the bioremediation hose (14, FIG. 3) close to the boat. When the pump on shore (15, FIG. 3) is turned on again, it produces enough pressure to push the one-way valve in the direction of the flow.

In a preferred alternative configuration, bioremediation product and air are fed to the boat from shore (42, FIG. 7). They would be fed from a double hose such as a welding hose. In a welding hose or similar double hose, both hoses are attached to each other (37, FIG. 6) and they can be fed independently. On shore, bioremediation product is fed from either a large container (16, FIG. 7) or a bioreactor (21, FIG. 7) to a double-hose reel on (29, FIG. 7) on shore. In order to stop the feed of the bioremediation product, the pump on shore (15, FIG. 7) is turned off. A one-way valve (43, FIG. 7) on the hose feeding the bioremediation product stops the feed. When more bioremediation product needs to be fed, the pump (15, FIG. 7) is turned on and the flow of the bioremediation product opens the one-way valve. In this preferred configuration, air is fed from the compressor (28, FIG. 7) also to the same double hose reel on shore (29, FIG. 7). From the double reel on shore, the double hose bioremediation hose (37, FIG. 6 and FIG. 7) brings to the boat bioremediation product in one line and air in the other. The double hose connects to swivels (32, FIG. 6 and FIG. 7) which are attached to two connector tubes (31, FIG. 6 and FIG. 7). From the bottom of the connector tubes, a double hose (40, FIG. 7) connects to a small double hose reel on the boat (36, FIG. 7). This reel lowers the application double-hose (38, FIG. 7) to the desired depth. The application hose line carrying air has a diffuser (33, FIG. 6 and FIG. 7) at its end. The small bubbles coming out from the diffuser mix and disperse the bioremediation product exiting from the other hose line which applies the bioremediation product. The air bubbles also provide oxygen for increased biological activity. The double-hose application hose carries a plastic weight (34, FIG. 6 and FIG. 7) to allow the hose to be fully extended. The depth required to lower the double hose is measured with the pressure sensor (8, FIG. 7) and the information is sent to shore via telemetry.

To reduce the drag of the weight of the bioremediation hose, hollow floats can be attached to the hose every few feet as the hose leaves the on shore reel. Hollow floating sticks (41, FIG. 6.) such as the ones used in swimming pools can be used. The floats can be of a color which makes them visible from far to see how the hose is extending. As the boat finishes its task and the hose is reeled back from shore, the floats can be removed as the hose is reeled back.

If more efficient oxygenation of the body of water is required, nanobubbles can be pumped from a nanobubble generator at shore. Nanobubbles are less than 200 nanometers in diameter and have no buoyancy. They are not lost to the atmosphere; instead, they remain in water until their oxygen content is utilized. Nanobubbles can be produced from air, pure oxygen or from ozone. Nanobubbles also have strong disinfecting power. If it is necessary to reduce pathogen numbers, cyanotoxins and algae as well as to break down cyanotoxins, then nanobubbles can be used. Nanobubbles ability to disinfect and break down cyanotoxins increases based on their composition. Air has the least disinfecting power, pure oxygen has stronger disinfection characteristics and ozone is the most powerful. Nanobubbles lose their disinfection capacity over time. Their disinfection power can be assessed with an oxidation-reduction probe such as the ones used by the apparatus in this patent application. Once the disinfection power has subsided, bioremediation microorganisms can be applied with the apparatus to outcompete the remaining coliforms and pathogens.

If the bioremediation products used for a body of water are microorganisms, they can be grown onsite with a bioreactor (21, FIG. 3 and FIG. 7)) The bioreactor such as in U.S. Pat. No. 10,981,818 B2 can produce large amounts of concentrated microorganisms. The microorganisms used for bioremediation are safe because they are non-pathogenic and non-genetically modified. The microorganisms are selected depending on what problems are present in the body of water. For example, Nitrosomonas and Nitrobacter nitrify excess ammonia converting it into nitrate. Another set of bacteria such as Pseudomonas, Thiobacillus and Paracoccus denitrify nitrate and produce nitrogen gas allowing the removal of nitrogen from water. Other bacteria oxidize hydrogen sulfide, mercaptans and other sulfur compounds controlling their toxicity and malodor. Various contaminants including organic toxicants can be eliminated by digestion using the selected microorganisms. Some microorganisms that can be used in bioremediation are bacteria, fungi, actinomyces, streptomyces, protozoa and metazoa. All these types of specialized microorganisms are known in the field of bioremediation and can be purchased from various suppliers such as Novozymes in Franklinton (N.C.), Sustainable Nutrition in Jefferson City (TN) and others. In the art of bioremediation, enzymes are also used. An enzyme is a protein capable of degrading a specific pollutant. If many different types of pollutants need to be degraded, a blend of enzymes can be used. Enzymes can be purchased from Novozymes, CEKAL in Mount Holly (NC) and multiple suppliers. Biostimulants are also used in the art of bioremediation. Their function is to enhance the ability of the biology in the body of water to improve pollutant degradation as well as to enhance the efficiency of bioremediation microorganisms that are applied. Biostimulants are based on micronutrients and plant extracts. If the body of water is deficient in macronutrients such as nitrogen and phosphorous and if this deficiency is not allowing the biology to digest contaminants, macronutrients can be applied. Sometimes, it may be necessary to add oxygen to a septic portion of the body of water to activate its biology. There are several chemicals that provide oxygen such as sodium perborate, calcium peroxide, magnesium peroxide, calcium nitrate, potassium nitrate and various nitrate containing chemicals. If there is a need to precipitate phosphates in the body of water to control eutrophication, a phosphate precipitant product can be used to make the phosphates biologically unavailable. Some of these chemicals are salts of cationic ions such as ferrous salts, ferric salts, aluminum salts and lanthanum salts. If the body of water is heavily contaminated with coliforms and various pathogens and if there is a need to control their numbers, nanobubbles can also be applied as part of the bioremediation process.

Although the apparatus and methods described in the patent application have been described with reference to the preferred embodiments, it will be understood by those skilled in the art of bioremediation that changes in form and detail may be made therein without departing from the spirit and scope of the invention.

We claim:

1. A remote control boat or similar apparatus for taking analytical readings of wastewater and contaminated recreational bodies of water, for taking samples of water and sludge and for delivering bioremediation products to the body of water comprising:
a power source; a remote controlled servo to control the speed of the motor; a remote controlled servo to control direction via rudders; various probes or a multi-probe unit with telemetry and visual interface to read and send data to shore of pH, temperature, conductivity, salinity, turbidity, total dissolved solids, dissolved oxygen, oxidation-reduction, ammonia, nitrate, phosphate as well as algae and chlorophyll levels; an on board GPS with telemetry to send data to shore of the location of the boat; an enclosure for the metering devices of the probes; a sonar with telemetry to measure and map sludge blanket thickness in wastewater lagoons, pond and contaminated bodies of water; remote controlled reels on the boat to lower probes, to lower devices to take water and sludge samples and to lower a double hose to apply bioremediation products and air; sampling devices for water and for sludge such as a bacon bomb, hose with a one-way valve or a bladder pump; a transmitter and receiver system to control the remote control elements of the apparatus with display; a single hose to bring from shore bioremediation product; a double hose to bring from shore bioremediation product and air; an application hose to apply bioremediation product to the body of water; a double hose application hose to apply bioremediation product in one line and air in the other so air mixes and disperses bioremediation product as it is being applied; a weight at the end of the probe cable, sampling devices and application hose to extend them while submerged; a pressure sensor with telemetry to measure the depth where probes, sampling devices or bioremediation hose needs to be submerged; a plate attachment on the boat to hold connector tubes to attach the application hose; swivels on the top of the connector tubes for the attachment of the bioremediation hose to the boat; a one-way valve on the bioremediation hose to stop flow when needed; floats to keep the bioremediation hose from sinking and causing drag on the boat; a remote controlled self-priming pump on the boat to apply bioremediation product from the hull of the boat via application hose; a pump on shore to apply bioremediation product from shore; a double-hose reel on shore to receive bioremediation product from a large tank or a bioreactor on shore as well as air from a compressor on shore to send bioremediation product and air to the boat via the bioremediation hose; a compressor on shore to supply air to the boat via remediation hose.

2. A remote control boat or similar apparatus for taking analytical readings of wastewater and contaminated recreational bodies of water, for taking samples of water and sludge and for delivering bioremediation products as in claim 1, wherein the body of water is a wastewater lagoon or pond or a contaminated recreational body of water.

3. A remote control boat or similar apparatus for taking analytical readings of wastewater and contaminated recreational bodies of water, for taking samples of water and sludge and for delivering bioremediation products as in claim 1, wherein remote controlled servos control the speed and direction of the boat.

4. A remote control boat or similar apparatus for taking analytical readings of wastewater and contaminated recreational bodies of water, for taking samples of water and sludge and for delivering bioremediation products as in claim 1, wherein said bioremediation products are selected from bacteria, fungi, actinomyces, streptomyces, enzymes, protozoa, metazoan, plant-based biostimulants, micronutrients, macronutrients, products to provide oxygen such as sodium perborate, calcium peroxide, magnesium peroxide, calcium nitrate, potassium nitrate, various nitrate containing chemicals, products to precipitate phosphorous such as aluminum salts, ferrous salts, ferric salts and lanthanum salts as well as nanobubbles to control coliforms, pathogens and algae.

5. A remote control boat or similar apparatus for taking analytical readings of wastewater and contaminated recreational bodies of water, for taking samples of water and sludge and for delivering bioremediation products as in claim 1, wherein the apparatus carries one or various single or double-hose remote control reels onboard to lower probes, sampling devices and hoses to apply bioremediation product and air.

6. A remote control boat or similar apparatus for taking analytical readings of wastewater and contaminated recreational bodies of water, for taking samples of water and sludge and for delivering bioremediation products as in claim 1, wherein the apparatus uses a weight to maintain probes, sampling devices and bioremediation application hoses fully submersed in the wastewater lagoon, pond or contaminated recreational body of water.

7. A remote control boat or similar apparatus for taking analytical readings of wastewater and contaminated recreational bodies of water, for taking samples of water and sludge and for delivering bioremediation products as in claim 1, wherein the apparatus uses a pressure sensor with telemetry to indicate the depth of the probes, sampling devices or bioremediation hose submersed in the wastewater lagoon, pond or contaminated recreational body of water.

8. A remote control boat or similar apparatus for taking analytical readings of wastewater and contaminated recreational bodies of water, for taking samples of water and sludge and for delivering bioremediation products as in claim 1, wherein the apparatus uses an array of probes or a multi-probe unit with telemetry capabilities and visual interface to measure and store or send data to shore of pH, temperature, conductivity, salinity, turbidity, total dissolved solids, dissolved oxygen, oxidation-reduction, ammonia, nitrate, phosphate as well as algae and chlorophyll levels to determine the health of a wastewater lagoon or pond or a contaminated recreational body of water.

9. A remote control boat or similar apparatus for taking analytical readings of wastewater and contaminated recreational bodies of water, for taking samples of water and sludge and for delivering bioremediation products as in claim 1, wherein the apparatus uses an onboard GPS system with telemetry to indicate the location of the boat and indicate the location of readings taken, samples collected and application of bioremediation.

10. A remote control boat or similar apparatus for taking analytical readings of wastewater and contaminated recreational bodies of water, for taking samples of water and sludge and for delivering bioremediation products as in claim 1, wherein the apparatus uses a sonar to measure and map the thickness of the sludge blanket at the bottom of wastewater lagoons, pond and contaminated recreational bodies of water.

11. A remote control boat or similar apparatus for taking analytical readings of wastewater and contaminated recreational bodies of water, for taking samples of water and sludge and for delivering bioremediation products as in claim 1, wherein the apparatus has the ability to take water samples of wastewater lagoons, ponds and contaminated recreational bodies of water using a bacon bomb, a hose with one-way check valve, a bladder pump, a modified sludge judge or other similar sampling device in order to do chemical and biological analysis to determine the health of the body of water.

12. A remote control boat or similar apparatus for taking analytical readings of wastewater and contaminated recreational bodies of water, for taking samples of water and sludge and for delivering bioremediation products as in claim 1, wherein the apparatus has the ability to take sludge samples of wastewater lagoons, ponds and contaminated recreational bodies of water using a bacon bomb, a hose with a one-way check valve, a bladder pump, a modified sludge judge or other similar sampling device to do chemical and biological analysis on the samples to determine the health of the body of water.

13. A remote control boat or similar apparatus for taking analytical readings of wastewater and contaminated recreational bodies of water, for taking samples of water and sludge and for delivering bioremediation products as in claim 1, wherein the apparatus uses a plate attachment on the boat to attach connector tubes for the bioremediation single or double hose and for the attachment of the application hose whether it is a single or double hose.

14. A remote control boat or similar apparatus for taking analytical readings of wastewater and contaminated recreational bodies of water, for taking samples of water and sludge and for delivering bioremediation products as in claim 1, wherein the apparatus uses swivels attached to the connector tubes with the swivel elements attaching to the bioremediation hose to reduce the chance that the bioremediation hose becomes tangled.

15. A remote control boat or similar apparatus for taking analytical readings of wastewater and contaminated recreational bodies of water, for taking samples of water and sludge and for delivering bioremediation products as in claim 1, wherein said bioremediation products are applied to the body of water from the hull of the boat using an onboard remote controlled self-priming pump and an application hose attached to the boat connector tubes.

16. A remote control boat or similar apparatus for taking analytical readings of wastewater and contaminated recreational bodies of water, for taking samples of water and sludge and for delivering bioremediation products as in claim 1, wherein said bioremediation products are applied to the body of water from the hull of the boat using an onboard remote-controlled pump and an application hose which is lowered to the desirable depth using a remote-controlled reel on the boat.

17. A remote control boat or similar apparatus for taking analytical readings of wastewater and contaminated recreational bodies of water, for taking samples of water and sludge and for delivering bioremediation products as in claim 1, wherein said bioremediation products are delivered to refill the hull of the boat from a reel on shore which said reel is fed the bioremediation product from a large container or an on-site bioreactor also on shore.

18. A remote control boat or similar apparatus for taking analytical readings of wastewater and contaminated recreational bodies of water, for taking samples of water and sludge and for delivering bioremediation products as in claim 1, wherein said bioremediation products are delivered from a reel on shore which said reel is fed the bioremediation product from a large container or an on-site bioreactor wherein bioremediation product is sent from said shore reel via a remediation hose with said hose attached to swivels on connector tubes on the boat and bioremediation product delivered from the application hose directly attached to the lower portion of the connector tubes.

19. A remote control boat or similar apparatus for taking analytical readings of wastewater and contaminated recreational bodies of water, for taking samples of water and sludge and for delivering bioremediation products as in claim 1, wherein said bioremediation products are delivered from a reel on shore which said reel is fed from a large container or an on-site bioreactor also on shore wherein bioremediation product is sent from said shore reel via a remediation hose with said hose attached to swivels on connector tubes of the boat and delivered to a remote controlled reel on the boat which lowers the application hose to apply said bioremediation product to the body of water.

20. A remote control boat or similar apparatus for taking analytical readings of wastewater and contaminated recreational bodies of water, for taking samples of water and sludge and for delivering bioremediation products as in claim 1, wherein said bioremediation products are delivered from a double-hose reel on shore which said reel is fed the bioremediation product in one line and air from a compressor in the second line with said bioremediation product and air fed to the boat via a double bioremediation hose that connects to swivels on the boat and then to a remote controlled double reel on the boat which lowers a double hose application hose to deliver bioremediation product and air to the body of water.

21. A remote control boat or similar apparatus for taking analytical readings of wastewater and contaminated recreational bodies of water, for taking samples of water and sludge and for delivering bioremediation products as in claim 1, wherein the bioremediation hose and the application hose are both double hoses with one line carrying bioremediation product and the other line carrying air.

22. A remote control boat or similar apparatus for taking analytical readings of wastewater and contaminated recreational bodies of water, for taking samples of water and sludge and for delivering bioremediation products as in claim 1, wherein the exit of the airline of the double-hose application hose has a diffuser to help mix and disperse the bioremediation product as it exits from the other hose line.

23. A remote control boat or similar apparatus for taking analytical readings of wastewater and contaminated recreational bodies of water, for taking samples of water and sludge and for delivering bioremediation products as in claim 1, with the single or double bioremediation hose carries floats attached to it to maintain the said hose from sinking and causing drag on the boat.
